(19) United States
(12) Patent Application Publication
(10) Pub. No.: US 2009/0257982 A1
(43) Pub. Date: Oct. 15, 2009

(54) MEDICAL DEVICE FOR TREATING DIABETES MELLITUS, OBESITY, CHRONIC FATIGUE, AGING, AND OTHER MEDICAL CONDITIONS BY UTILIZING MODIFIED VIRUS VIRIONS TO INSERT MESSENGER RIBONUCLEIC ACID MOLECULES INTO CELLS

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 12/101,934

(22) Filed: Apr. 11, 2008

(51) Int. Cl. A61K 35/76; A61P 3/10

(52) U.S. Cl. 424/93.2

(57) ABSTRACT

The common link between diabetes mellitus, obesity, chronic fatigue and even aging may be related to deficiencies involving a body's metabolism of glucose and the ability to optimally conduct the necessary biologic processes of aerobic respiration. Utilizing a medical device comprised of a modified form of virus to deliver to cells in the body the messenger RNA molecules needed to construct insulin receptors and generate the enzymes that participate in the processes of glycolysis, the tricarboxylic acid cycle, oxidative phosphorylation and anaerobic respiration will lead to greater utilization of blood glucose and a more efficient and sustained production of the energy molecules that fuel the metabolic processes of the cell. Greater utilization of blood glucose will significantly advance the effort to correct the medical problems associated with diabetes, obesity, chronic fatigue, and aging.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2008 Lane B. Scheiber and Lane B. Scheiber II. A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device intended to correct a protein deficiency in the body by increasing the intracellular production of the deficient protein by utilizing a modified virus to insert one or more messenger ribonucleic acid molecules into one or more cells of the body.

2. Description of Background Art

Diabetes and obesity represent two very widespread and extremely important health issues across the United States. Both conditions affect a significant portion of the population and result in significant morbidity and mortality. In the United States, about 16 million people suffer from diabetes mellitus. Every year, about 650,000 additional people are diagnosed with the disease. Diabetes mellitus is the seventh leading cause of all deaths. According to the National Institutes of Health, in the United States, 97 million adults are overweight or obese. About 22.5 percent of the population is obese, up from 13 percent in 1960. Obesity and diabetes mellitus are linked disorders in many patients. Studies show that individuals who are 20 percent or more overweight run a greater risk of developing diabetes mellitus, hypertension, coronary heart disease, stroke, arthritis, and some forms of cancer.

Chronic fatigue syndrome (CFS) is characterized by extreme fatigue that has persisted in excess of six months. CFS occurs most commonly in people in their 40s and 50s. CFS is estimated to affect one million people in the United States, with estimated tens of millions with similar symptoms of fatigue that do not meet the strict criteria to make a formal diagnosis. CFS results in significant loss of productivity by the individuals affected by this condition, thus having a negative impact on the work-force in general.

Aging affects the entire scope of the population. The characteristics of aging may be related to a lack of sufficient sustained energy production and therefore a progressive decline in an individual's cellular capacity to sufficiently fuel biologic processes requiring energy. The mitochondria are the powerhouses of the cells, converting sugars such as glucose to energy molecules such as adenosine triphosphate (ATP). A liver cell may contain as many as 2500 mitochondria. If the mitochondria lack a sufficient amount of any of the required enzymes to complete the process of metabolizing glucose to energy molecules such as ATP, necessary biologic functions may not be available to engage in cellular functions as needed. It has been estimated that in a person's lifetime, between the ages of 35 to 50 years old, there occurs a fifty percent decline in the mitochondria's capacity to produce energy. As an individual ages, there is a further decline in mitochondrial output capacity.

Diabetes mellitus, obesity, chronic fatigue, and aging may all be linked to the vital process of glucose metabolism. Providing the body with the means of maintaining an optimum level of glucose metabolism and anaerobic respiration may provide the means to effectively manage the above-mentioned four major health conditions.

Diabetes mellitus represents a state of hyperglycemia, which is generally defined as a serum blood sugar that is higher than what is considered the normal range for humans. Glucose, a six-carbon molecule, represents a form of sugar. Glucose is absorbed by the cells of the body through passive diffusion and is converted to energy by the biologic processes of glycolysis, the tricarboxylic acid (TCA) cycle and oxidative phosphorylation. Insulin, a protein, facilitates the absorption of glucose into cells. Normal range for blood glucose in humans is generally defined as a fasting blood plasma glucose level of between 70 to 110 mg/dl. An alternative means of measuring the blood glucose level is by measuring the glucose level stored inside red blood cells in the blood, which provides information regarding the average blood glucose spanning several weeks, rather than a real-time blood glucose level represented in a blood plasma glucose level. Measurement of glycated hemoglobin (Hgb A1C) provides an estimate of diabetic control over the preceding three months, with a normal value for nondiabetic patients being approximately 6% and values for a poorly controlled diabetic being 7-12%. For purposes of this description the terms “plasma’ and ‘serum’ refer to the same substance, which is the fluid (noncellular) portion of blood.

Diabetes mellitus is generally classified as Type One and Type Two. Type One diabetes mellitus is insulin dependent, and refers to the condition where there is an insufficient quantity of insulin molecules circulating in the blood stream compared to the quantity required to maintain the blood glucose level within the recognized normal range. In Type One diabetes mellitus insulin must be provided to the body in order to properly regulate the blood plasma glucose level. When insulin is required to regulate the blood glucose level in the body, this condition is often referred to as insulin dependent diabetes mellitus (IDDM). Type Two diabetes mellitus is not dependent upon insulin and is often referred to as noninsulin dependent diabetes mellitus (NIDDM), meaning the blood glucose level can be managed without treatment with exogenous insulin, and is generally managed by means of diet, exercise or intervention with oral medications. Type Two diabetes mellitus is considered a progressive disease, with the underlying pathogenic mechanisms including pancreatic beta cell (often designated as β-Cell) dysfunction and insulin resistance.

The pancreas serves as an endocrine gland and an exocrine gland. Functioning as an endocrine gland, the pancreas produces and secretes hormones including the hormones insulin and glucagon. Insulin acts to reduce levels of glucose circulating in the blood. Beta cells secrete insulin into the blood when a higher than normal level of glucose is detected in the serum. Glucagon is an antagonistic hormone to insulin, which acts to stimulate an increase in glucose circulating in the blood. Alpha cells in the pancreas secrete glucagon into the blood when a low level of glucose is detected in the blood.

Glucose generally enters the body and then the blood stream as a result of the digestion of food. For purposes of this description, ‘blood’ and ‘blood stream’ refer to the same substance, which is generally considered to be blood as a whole including plasma and blood cells. The beta cells of the Islets of Langerhans continuously sense the level of glucose in the blood and respond to elevated levels of blood glucose by secreting insulin into the blood. Beta cells produce the protein ‘insulin’ in the endoplasmic reticulum and store the insulin in vacuoles until the insulin is needed. When beta cells detect an increase in the glucose level in the blood, the beta cells release insulin into the blood plasma from said storage vacuoles.

Insulin is a protein. An insulin molecule consists of two chains of amino acids, an alpha chain and a beta chain, linked by two disulfide (S-S) bridges. The alpha chain consists of 21 amino acids. The beta chain consists of 30 amino acids.

Insulin interacts with the cells of the body by means of a cell-surface receptor termed the ‘insulin receptor’ located on the exterior of a cell's ‘outer membrane’, often referred to as the ‘plasma membrane’. Insulin interacts with muscle and liver cells by means of the insulin receptor to rapidly remove excess blood sugar when the glucose level in the blood is higher than the upper limit of the normal physiologic range. Recognized functions of insulin include stimulating cells to take up glucose from the blood, convert glucose to glycogen (an extensively branched glucose storage polysaccharide molecule), to facilitate the cells in the body to utilize glucose to generate biochemically usable energy, and to stimulate fat cells to take up glucose and synthesize fat.

Diabetes Mellitus may develop in an individual as the result of one or more factors. Causes of diabetes mellitus may include: (1) mutation of the insulin gene itself causing miscoding, which results in the production of ineffective insulin molecules; (2) mutations to genes that code for the ‘transcription factors’ needed for transcription of the insulin gene in the DNA to create messenger RNAs which facilitates the manufacture of the insulin molecule; (3) mutations of the gene encoding for the insulin receptor, which produces inactive or an insufficient number of insulin receptors; (4) mutation to the gene encoding for glucokinase, the enzyme that phosphorylates glucose in the first step of glycolysis; (5) mutations to the genes encoding portions of the potassium channels in the plasma membrane of the beta cells, preventing proper closure of the channel, thus blocking insulin release; (6) mutations to mitochondrial genes that as a result, decreases the energy available to be used facilitate the release of insulin, therefore reducing insulin secretion; (7) failure of glucose transporters to properly permit the facilitated diffusion of glucose from plasma into the cells of the body.

A eukaryote refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure cell-surface receptors that the cell uses to detect properties of the environment surrounding the cell membrane. Cytoplasm refers to the entire contents inside the cell except for the nucleus and acts as a filling medium inside the boundaries of the plasma cell membrane. Cytosol refers to the semifluid portion of the cytoplasm minus the mitochondria and the endoplasmic reticulum. The nucleus, organelles, and ribosomes are suspended in the cytosol. Nutrients such as amino acids, oxygen and glucose are present in the cytosol. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex. Floating in the cytoplasm, but also located in the endoplasmic reticulum and mitochondria are ribosomes. Ribosomes are protein structures comprised of several strands of proteins that combine and couple to a messenger ribonucleic acid (mRNA) molecule. More than one ribosome may be attached to a single mRNA at a time. Ribosomes decode genetic information coded in a mRNA molecule and manufacture proteins to the specifications of the instruction code physically present in the mRNA molecule.

The majority of the deoxyribonucleic acid (DNA) in a cell is present in the form of chromosomes, the double stranded helical structures located in the nucleus of the cell. DNA in a circular form, can also be found in the mitochondria, the powerhouse of the cell, an organelle that assists in converting glucose into usable energy molecules. DNA represents the genetic information a cell needs to manufacture the materials it requires to develop to its mature form, sustain life and to replicate. Genetic information is stored in the DNA by arrangements of four nucleotides referred to as: adenine, thymine, guanine and cytosine. DNA represents instruction coding, that in the process known as transcription, the DNA's genetic information is decoded by transcription protein complexes referred to as polymerases, to produce ribonucleic acid (RNA). RNA is a single strand of genetic information comprised of coded arrangements of four nucleotides: adenine, uracil, guanine and cytosine. The physical difference in the construction of a DNA molecule versus a RNA molecule is that DNA utilizes the nucleotide ‘thymine’, while RNA molecules utilize the nucleotide ‘uracil’. RNAs are generally classified as messenger RNAs (mRNA), transport RNAs (tRNA) and ribosomal RNAs (rRNA).

Mitochondrion (‘mitochondria’ pleural) is a cellular organelle that is considered the energy producing organelle of the cell. Mitochondria assist in generating energy for cell metabolism by producing ATP molecules from glucose. Within the cytoplasm and outer wall of the mitochondria sugar molecules undergo the process of glycolysis, and then inside the mitochondria byproducts of glucose are further broken down in the tricarboxylic acid (TCA) cycle and by oxidative phosphorylation to produce useable forms of energy molecules.

The exterior of a mitochondrion is known as an external membrane. Inside the outer membrane is an inner membrane. Folds in the inner membrane create crista, which expands the surface of the inner membrane and enhances the mitochondrion's ability to create ATP molecules. Inside the inner membrane is the mitochondrion matrix. The mitochondrion matrix contains a highly concentrated mixture of enzymes, ribosomes, tRNA and mitochondrial DNA. Glycolysis occurs in the cytosol of the cell and membrane of the mitochondrion. The tricarboxylic acid cycle functions within the inner chambers and matrix of the mitochondrion. Oxidative phosphorylation occurs within the boundaries of the outer and inner membranes of the mitochondrion.

Many of the intermediates of the processes of glycolysis and the tricarboxylic acid cycle exist as anions at the pH found in cells, and readily associate with H⁺ to form acids. The intermediates of glycolysis and the tricarboxylic acid cycle are therefore often written as either an anion or an acid. For the purposes of this description, the intermediates of the processes of glycolysis and the tricarboxylic acid cycle are generally written as anions (as an example pyruvate versus pyruvic acid).

As a result of the biochemical process of glycolysis during aerobic (oxygen available) respiration conditions glucose is converted to pyruvate. The abbreviated processes of glycolysis include: (1) Glucose is converted to glucose-6-phosphate by the enzyme ‘hexokinase’. (2) Glucose-6-phosphate is converted to fructose-6-phosphate by the enzyme ‘glucose-6-phosphate isomerase’. (3) Fructose-6-phosphate is converted to fructose 1,6-diphosphate by the enzyme ‘phosphofructo kinase’. (4) Fructose 1,6-diphosphate is converted to two different entities including dihydroxyacetone-3-phosphate and glyceraldehydes-3-phosphate by the enzyme ‘fructose bisphosphate aldolase’. (5) Dihydroacetone-3-phosphate converts to D-glyceraladehyde-3-phosphate by the enzyme ‘triose-phosphate isomerase’. (6) Glyceraldehyde-3-phosphate is converted to 1,3-diphosphoglycerate by the enzyme ‘glyceraldehyde-3-phosphate dehydrogenase’. (7) 1,3-diphosphoglycerate is converted to 3-phosphoglycerate by the enzyme ‘phosphoglycerate kinase’. (8) 3-phosphoglycerate is converted to 2-phosphoglycerate by the enzyme ‘phosphoglycerate mutase’. (9) 2-phosphoglycerate is converted to phosphoenolpyruvate by the enzyme ‘enolase’. (10) Phosphoenolpyruvate is converted to pyruvate by the enzyme complex referred to as ‘pyruvate kinase’.

Pyruvate is then oxidized to an acetyl group, which is combined with Coenzyme A and produces acetyl Coenzyme A (acetyl-CoA). Pyruvate dehydrogenase, which metabolizes pyruvate to acetyl-CoA is comprised of a multi-enzyme complex. The three protein complexes of pyruvate dehydrogenase are designated E1 (pyruvate dehydrogenase), E2 (dihydrolipoamide S-acetyltransferase), and E3 (dihydrolipoamide dehydrogenase). Acetyl-CoA enters the tricarboxylic acid cycle. Under aerobic respiration conditions from one glucose molecule the process of glycolysis generates 8 ATP molecules, conversion of pyruvate to acetyl CoA generates an additional 6 ATP molecules.

The tricarboxylic acid cycle otherwise known as the citric acid cycle or the Krebs cycle, was discovered in 1937 by Sir Hans Krebs, and is a biochemical process that provides complete oxidation of acetyl-CoA, which may be derived from sources such as fats, carbohydrates and lipids. For purposes of this discussion, acetyl-CoA is a byproduct of glucose metabolism during glycolysis, and enters the tricarboxylic acid cycle and (1) combines with oxaloacetate (also known as oxaloacetic acid) by the action of the enzyme ‘citrate synthetase’ which produces citrate (also known as citric acid). (2) Citrate is converted to cis-aconitate per the enzyme ‘aconitase’. (3) Cis-aconitate is converted to iso-citrate (also known as isocitur acid) again by the enzyme aconitase. (4) Iso-citrate is converted to alpha-ketoglutarate by the enzyme ‘isocitrate dehydrogenase’. (5) Alpha-ketoglutaric acid is converted to succinyl CoA by the enzyme ‘2-oxoglutarate dehydrogenase’. (6) Succinyl CoA is converted to succinate (also known as succinic acid) by the enzyme ‘succinyl-CoA synthetase’. (7) Succinate is converted to fumarate (also known as fumaric acid) by the enzyme ‘succinate dehydrogenase’. (8) Fumarate is converted to malate (also known as malic acid) by the enzyme ‘fumarate hydratase’. (9) Malate is converted to oxaloacetate by the enzyme ‘malate dehydrogenase’. The result of metabolism of glucose by glycolysis and the tricarboxylic acid cycle yields ATP molecules and electron donor molecules such as the reduced form of the coenzyme nicotinamide adenine dinucleotide written NADH+H⁺. The tricarboxylic acid cycle also produces electron donor molecules in the form of the reduced co-enzyme flavin adenine dinucleotide written FADH₂.

Oxidative phosphorylation is a metabolic pathway that uses energy released by oxidation to produce adenosine triphosphate (ATP). During oxidative phosphorylation electrons are transferred from electron donors to electron acceptors such as oxygen in redox reactions. In eukaryotes the redox reactions are carried out by a series of protein complexes located within mitochondria. These protein complexes represent a linked set of enzymes referred to as electron transport chains. The protein complexes utilized in oxidative phosphorylation include nicotinamide adenine dinucleotide (NADH) dehydrogenase enzyme molecule, the succinate dehydrogenase enzyme molecule, the cytochrome-c reductase enzyme molecule, the cytochrome-c oxidase enzyme molecule, and the ATP synthase enzyme molecule. Under aerobic respiration conditions one glucose molecule metabolized by the combination of glycolysis, the tricarboxylic acid cycle and oxidative phosphorylation yields as many as 38 ATP molecules.

An enzyme is a protein generated by cells that acts as a catalyst to induce chemical changes in other substances, itself remaining apparently unchanged in the process. There are several main groups of enzymes including oxidoreductase, transferase, hydrolase, lyase, isomerase, and ligase, sometimes referred to as synthetase. EC is an abbreviation for Enzyme Commission of the International Union of Biochemistry and this is used in conjunction with a unique number to define a specific enzyme identified in the Enzyme Commission's list of enzymes. Oxidoreductases generally have as their first EC identifying number, the number 1. Transferases generally have as their first EC identifying number, the number 2. Hydrolases generally have as their first EC identifying number, the number 3. Lyases generally have as their first EC identifying number, the number 4. Isomerase generally have as their first EC identifying number, the number 5. Ligases generally have as their first EC identifying number, the number 6. Several scientific names often exist to identify the same enzyme.

Enzymes (followed by their Enzyme Commission of the International Union of Biochemistry number) utilized in the metabolism of glucose in the processes of glycolysis, tricarboxylic acid cycle, and oxidative phosphorylation are described in the following paragraphs.

Hexokinase (EC 2.7.1.1), is also referred to as hexokinase type IV glucokinase or in some cases simply glucokinase. Hexokinase converts glucose to glucose-6-phosphate in glycolysis.

Glucose-6-phosphate isomerase (EC 5.3.1.9), is also known as phosphoglucoisomerase. Glucose-6-phosphate isomerase is an enzyme that converts glucose-6-phosphate to fructose-6-phosphate in glycolysis.

6-phosphofructokinase (EC 2.7.1.11), is also known as phosphofructokinase. 6-phosphofructokinase is an enzyme that converts fructose-6-phosphate to fructose 1,6-diphosphate in glycolysis.

Fructose bisphosphate aldolase (EC 4.1.2.13), is also known as aldolase. Fructose bisphosphate aldolase is an enzyme that converts fructose 1,6-diphosphate to two different entities including dihydroxyacetone 3-phosphate and glyceraldehydes 3-phosphate in glycolysis.

Triose-phosphate isomerase (EC 5.3.1.1). Triose-phosphate isomerase is an enzyme that converts dihydroacetone-3-phosphate converts to D-glyceraladehyde-3-phosphate.

Glyceraldehyde-3-phosphate dehydrogenase (EC 1.2.1.12), may be abbreviated GAPDH or G3PDH. Glyceraldehyde-3-phosphate dehydrogenase is an enzyme that converts glyceraldehydes-3-phosphatate to 1,3-diphosphoglycerate in glycolysis.

Phosphoglycerate kinase (EC 2.7.2.3). Phosphoglycerate kinase is an enzyme that converts 1,3-diphosphoglycerate to 3-phosphoglycerate in glycolysis.

Phosphoglycerate mutase (EC 5.4.2.1). Phosphoglycerate mutase is an enzyme that converts 3-phosphoglycerate to 2-phosphoglycerate in glycolysis.

Enolase (EC 4.2.1.11). Enolase is an enzyme that converts 2-phosphoglycerate to phosphoenolpyruvate in glycolysis.

Pyruvate kinase (EC 2.7.1.40). Pyruvate kinase is an enzyme that converts phosphoenolpyruvate to pyruvate in glycolysis.

Pyruvate dehydrogenase is comprised of three units. The three units include E1 (EC 1.2.4.1), (EC 1.2.1.51), E2 dihydrolipoamide S-acetyltransferase (EC 2.3.1.12), and E3 dihydrolipoamide dehydrogenase (EC 1.8.1.4). Pyruvate dehydrogenase molecular complex catalyzes the conversion of pyruvate to acetyl-CoA.

Citrate synthetase (EC 4.1.3.7). Citrate synthetase is an enzyme that converts acetyl-CoA combines with oxaloacetate to produce citrate in the tricarboxylic acid cycle.

Aconitase (EC 4.2.1.3). Aconitase exists in two isoenzyme forms in eukaryotes: mitochondrial and cytosolic. Aconitase is an enzyme that converts citrate to cis-aconitate in the Tricarboxylic acid cycle and converts cis-aconitate to iso-citrate in the tricarboxylic acid cycle.

Isocitrate dehydrogenase (EC 1.1.1.41). Isocitrate dehydrogenase is an enzyme that converts isocitrate to alpha-ketoglutaric acid in the tricarboxylic acid cycle.

2-oxoglutarate dehydrogenase is a protein complex comprised of three units. The three units include E1 (EC 1.2.4.2), E2 (EC 2.3.1.61), and E3 (EC 1.8.1.4). 2-oxoglutarate dehydrogenase is an enzyme complex that converts alpha-ketoglutaric to succinyl CoA in the tricarboxylic acid cycle.

Succinyl-CoA synthetase (EC 6.2.1.5). Succinyl-CoA synthetase is an enzyme that converts succinyl CoA to succinate in the tricarboxylic acid cycle.

Succinate dehydrogenase (EC 1.3.5.1). Succinate dehydrogenase is an enzyme that converts succinate to fumarate in the tricarboxylic acid cycle.

Fumarate hydratase (EC 4.2.1.2). Fumarate hydratase is an enzyme that converts fumarate to malate in the tricarboxylic acid cycle.

Malate dehydrogenase (EC 1.1.1.37). Malate dehydrogenase is an enzyme that converts malate to oxaloacetate in the tricarboxylic acid cycle.

During conditions were sufficient oxygen is available, metabolism of glucose generally occurs through the processes of glycolysis, tricarboxylic acid cycle, and oxidative phosphorylation. When oxygen is not readably available, anaerobic respiration occurs. The enzyme lactate dehydrogenase provides an alternative pathway to produce ATP when sufficient oxygen is not available by converting pyruvate to lactic acid. The anaerobic pathway lactate dehydrogenase catalyzes is much less efficient means of producing energy molecules than the aerobic pathway that takes advantage of oxygen dependent processes of tricarboxylic acid cycle and oxidative phosphorylation.

Proteins are comprised of a series of amino acids bonded together in a linear strand, sometimes referred to as a chain; a protein may be further modified to be a structure comprised of one or more similar or differing strands of amino acids bonded together. A protein comprised of more than one strand of amino acids (referred to as subunits) may be referred to as a protein complex. Insulin is a protein structure comprised of two strands of amino acids, one strand comprised of 21 amino acids long and the second strand comprised of 30 amino acids; the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors.

Messenger RNAs (mRNA) are created by transcription of DNA. Messenger RNA generated by transcription of nuclear DNA, migrate out of the nucleus of the cell, and are utilized as protein manufacturing templates by ribosomes. Different mRNAs code for different proteins. As previously mentioned, there are as many as 30,000 varieties of proteins, therefore there are at least 30,000 different mRNA molecules. A ribosome is a protein complex that manufactures proteins by deciphering the instruction code located in a mRNA molecule. When a specific protein is needed, pieces of the ribosome complex bind around the strand of mRNA that carries the specific instruction code that will generate the required protein. The ribosome traverses the mRNA strand and deciphers the genetic information coded into the sequence of nucleotides that comprise the mRNA molecule.

Transport RNAs (tRNA) are constructed in the nucleus or in the mitochondria, and are coded for one of the 20 amino acids the cells of the human body use to construct proteins. Once a tRNA is created by transcription of the DNA, the tRNA seeks out the type of amino acid it has been coded for and attaches to that specific amino acid. The tRNA then delivers the amino acid it carries to a ribosome that is waiting for that specific amino acid. Proteins are manufactured by the ribosomes binding together sequences of amino acids. The order by which the amino acids are bonded together is dictated by the way the mRNA is constructed and how the ribosome interprets the information encoded in the string of nucleotides present in the mRNA strand.

A sequence of three nucleotides present in a mRNA molecule represents a unit of information referred to as a codon. Codons code for all of the 20 amino acids used to construct protein molecules and also for START and STOP commands. In the process known as translation, the ribosome decodes the codons present in the mRNA, initiating the protein manufacturing process at a START codon, then interfacing with tRNAs carrying the amino acids that match the sequence of codons in the mRNA as the ribosome traverses the length of the mRNA molecule. The ribosome functions as a protein factory by taking amino acids delivered by tRNAs and binding the amino acids together in the order dictated by the sequence of codon instructions coded into the mRNA template as directed by the manner of the nucleic acid arrangement in the mRNA molecule. Protein synthesis ceases when a ribosome encounters a STOP code. Once complete, the protein molecule is released by the ribosome.

The insulin molecule is a protein produced by beta cells located in the pancreas. The ‘insulin messenger RNA’ is created in a cell by transcribing the insulin gene from nuclear DNA in the nucleus of the cell. The native messenger RNA (mRNA) for insulin then travels to the endoplasmic reticulum, where numerous ribosomes engage these mRNA molecules. Many ribosomes may be attached to a single strand of mRNA simultaneously, each generating an identical copy of the protein as dictated by the information encoded in the mRNA. Insulin is produced by ribosomes translating the information in a mRNA molecule coded for the insulin protein, which produce strands of amino acids that are coded for an immature form of the biologically active insulin molecule referred to as ‘pro-insulin’. Once the pro-insulin molecule is generated it then undergoes modification by several enzymes including prohormone convertase one (PC1), prohormone convertase two (PC2) and carboxypeptidase E, which results in the production of a biologically active insulin molecule. Once the biologically active insulin protein is generated, it is stored in a vacuole in the beta cell to await being released into the blood stream.

The insulin receptor, prohormone convertase one (PC1), prohormone convertase two (PC2) and carboxypeptidase E are produced in a similar fashion as to how pro-insulin and insulin are produced in a beta cell. A messenger RNA is transcribed from DNA, specific for either the insulin receptor, prohormone convertase one (PC1), prohormone convertase two (PC2) or carboxypeptidase E. When a messenger RNA coded for an insulin receptor is present and available, ribosomes will attach to the mRNA and generate insulin receptor proteins. When a messenger RNA coded for either prohormone convertase one (PC1), prohormone convertase two (PC2) or carboxypeptidase E is present and available, ribosomes will attach to the mRNA, decode the instructions in the mRNA molecule and generate the protein.

Insulin receptors, which appear on the surface of cells, offer binding sites for insulin circulating in the blood. When insulin binds to an insulin receptor, the biologic response inside the cell causes glucose to undergo processing in the cytoplasm. Processed glucose molecules then enter the mitochondrion. The mitochondrion further processes the modified glucose molecules to produce usable energy in the form of adenosine triphosphate molecules (ATP). Thirty-eight ATP molecules may be generated from one molecule of glucose during the process of aerobic respiration. ATP molecules are utilized as an energy source by biologic processes throughout the cell.

The current medical therapeutic approach to the management of diabetes mellitus has produced limited results. Patients with diabetes generally struggle with an inadequate production of insulin, or an ineffective release of biologically active insulin molecules, or a release of an insufficient number of biologically active insulin molecules, or an insufficient production of cell-surface receptors, or a production of ineffective cell-surface receptors, or a production of ineffective insulin molecules that are unable to interact properly with insulin receptors on cells to produce the required biologic effect. Type One diabetes requires administration of exogenous insulin. The traditional approach to Type Two diabetes has generally first been to adjust the diet to limit the caloric intake the individual consumes. Exercise is used as an initial approach to both Type One and Type Two diabetes as a means of up-regulating the utilization of fats and sugar so as to reduce the amount of circulating plasma glucose. When diet and exercise are inadequate in properly managing Type Two diabetes, oral medications are often introduced. The action of sulfonylureas, a commonly prescribed class of oral medication, is to stimulate the beta cells to produce additional insulin receptors and enhance the insulin receptors' response to insulin. Biguanides, another form of oral treatment, inhibit gluconeogenesis, the production of glucose in the liver, thereby attempting to reduce plasma glucose levels. Thiazolidinediones (TZDs) lower blood sugar levels by activating peroxisome proliferator-activated receptor gamma (PPAR-γ), a transcription factor, which when activated regulates the activity of various target genes, particularly ones involved in glucose and lipid metabolism. If diet, exercise and oral medications do not produce a satisfactory control of the level of blood glucose in a diabetic patient, exogenous insulin is injected into the body in an effort to normalize the amount of glucose present in the serum. Insulin, a protein, has not successfully been made available as an oral medication to date due to the fact that proteins in general become degraded when they encounter the acid environment present in the stomach.

Despite strict monitoring of blood glucose and potentially multiple doses of insulin injected throughout the day sometimes augmented with oral medications, many patients with diabetes mellitus still experience devastating adverse effects from elevated blood glucose levels. Microvascular damage and elevated tissue sugar levels contribute to such complications as renal failure, retinopathy involving the eyes, neuropathy, and accelerated heart disease despite aggressive efforts to maintain the blood sugar within the physiologic normal range using exogenous insulin by itself or a combination of exogenous insulin and one or more oral medications. Diabetes remains the number one cause of renal failure in this country. Especially in diabetic patients that are dependent upon administering exogenous insulin into their body, though dosing of the insulin may be four or more times a day and even though this may produce adequate control of the blood glucose level to prevent the clinical symptoms of hyperglycemia; this does not unerringly supplement the body's natural capacity to monitor the blood sugar level minute to minute, twenty-four hours a day, and deliver an immediate response to a rise in blood glucose by the release of insulin from beta cells as required. The deleterious effects of diabetes may still evolve despite strict and persistent exogenous control of the glucose level in the blood stream.

The strategy to treat obesity has been founded in encouraging the overweight individual to diet and exercise. When the traditional approach does not suffice and the patient is severely overweight, the strategy may take the form of surgically altering the size of the stomach to physically limit the consumption of food. All three approaches may be successful, but often there exists restrictions, which limit the use or success of these strategies.

Both obesity and weight related diabetes are to some effect related to a decline in the metabolism in the body, as a person ages. As mentioned previously, it has been identified that in individuals between the ages of 35 to 50 years old, the energy production generated by the mitochondria in human cells declines by 50%. Between the ages of 65 to 90 years old, energy production by the mitochondria declines by another 39%. Certainly if the energy supplied to human cells is reduced by such drastic percentages, this in part contributes to a decline in the overall metabolic rate of the cell and the body in general.

If a patient consumes a diet comprised of relatively the same amount of calories during their youth as they do in their middle age years and the utilization of the consumed sugar declines due to a significant decrease in the function of the mitochondria, the body has no alternative but to store the excess sugar and convert it to fat, thus resulting in the medical state of obesity. When the serum glucose level exceeds the upper limit for the normal range due to both a lack of adequate insulin production and a decline in glucose utilization by the mitochondria, diabetes emerges as a result.

The current treatment of diabetes may be augmented by the unique approach to utilizing modified viruses as vehicles to transport biologically active messenger ribonucleic acids (mRNA) coded to facilitate the manufacture of insulin receptors and the enzymes required to conduct the processes of glycolysis, the tricarboxylic acid cycle, oxidative phosphorylation and anaerobic respiration.

Viruses are obligate parasites. Viruses simply represent a carrier of genetic material and by themselves viruses are unable to replicate or carry on any form of biologic function outside their host cell. Viruses are generally comprised of one or more shells constructed of one or more layers of protein or lipid material, and inside the outer shell or shells, a virus carries a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell is a cell that provides the virus the proper biochemical machinery for the virus to successfully replicate itself.

Protected by the outer coat generally comprised of an envelope or capsid or envelope and capsid, viruses carry a genetic payload in the form of deoxyribonucleic acid (DNA) or ribonucleic acid (RNA). Once a virus's exterior probes locate and functionally engage the surface receptor or receptors on a host cell, the virus inserts its genetic payload into the interior of the host cell. In the event a virus is carrying a DNA payload, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate their RNA being converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the host cell's nuclear DNA and simply utilize portions of its innate viral genome to act as messenger RNA (mRNA). RNA viruses that bypass the host cell's DNA, cause the cell, in general, to generate copies of the necessary parts of the virus directly from the virus's RNA genome. When a virus's genome directly acts as a template, then similar to the cell's messenger RNA, the virus's RNA is read by the cell's ribosomes and proteins necessary to complete the virus's replication process are generated.

The Hepatitis C virus (HCV) is a positive sense RNA virus, meaning a type of RNA that is capable of bypassing the need for involving the host cell's nucleus by having its RNA genome function as messenger RNA. Hepatitis C virus infects liver cells. The Hepatitis C viral genome becomes divided once it gains access to the interior of a liver host cell. Portions of the subdivisions of the Hepatitis C viral genome directly interact with host liver cell's ribosomes to produce the proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to activate the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construct copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus offers a naturally occurring vehicle mechanism to transport and insert medically therapeutic messenger ribonucleic acid (mRNA) molecules into liver cells and other specifically targeted cells of the human body. The naturally occurring Hepatitis C virus already is equipped with the means of seeking out liver cells and delivering to liver cells its genetic payload. Further, the surface probes present on the Hepatitis C virus's outer protein coat can be modified to seek out specific receptors on specific target cells. Once the modified Hepatitis C virus's probes properly engage the cell-surface receptors on a target cell, the modified Hepatitis C virus would insert into the target cell one or more medically therapeutic mRNAs for the purpose of having the target cell generate proteins to achieve a medical therapeutic response.

The Hepatitis C virus is one of several viruses that have been identified that possess the natural capacity to locate and infect liver cells with the genome the virus carries, thus including a liver cell as part of its reproductive cycle. Hepatitis A virus (HAV), Hepatitis C virus (HCV), Hepatitis D virus (HDV), Hepatitis E virus (HEV), and Hepatitis G virus (HGV) have been identified to carry their genome as RNA. The Hepatitis G virus is considered to be very similar to the Hepatitis C virus. The Hepatitis F virus and Hepatitis H viruses at this point are not considered to exist, though this is controversial. The Hepatitis B virus (HBV) is believed to carry its genome as DNA. These alternative hepatitis viruses may also be utilized to act as alternative vehicles to deliver medically therapeutic messenger RNA molecules to liver cells or specific target cells.

Current state of gene therapy generally refers to efforts directed toward inserting an exogenous subunit of DNA into a vehicle such as a virus. The vehicle is intended to insert the exogenous subunit of DNA into a target cell. The exogenous DNA subunit then migrates to the target cell's nucleus. The exogenous DNA subunit then inserts into the native DNA of the cell. This represents a permanent alteration of the cell's nuclear DNA. At some point, the nuclear transcription proteins read the exogenous DNA subunit's nucleotide coding to produce the intended cellular response. The medical device described within the scope of this text involves RNA versus DNA as a modified virus virion's payload. DNA is comprised of the nucleotides adenine, thymine, guanine and cytosine. RNA is composed of the nucleotides adenine, uracil, guanine and cytosine. DNA codes for the manufacture of RNAs, which are composed of nucleotides. RNA codes for the manufacture of proteins, which are composed of amino acids. The virus chosen as the example of a the transport vehicle, Hepatitis C virus, is a RNA virus versus a virus that naturally carries a DNA genome.

It would seem an important element involved in the development of obesity, diabetes, lack of energy or reduced metabolism is linked to the failure of the mitochondria to properly utilize glucose to produce energy in the form of ATP.

The proteins manufactured by the ribosomes participate in the chemical reactions of glycolysis, the tricarboxylic acid cycle, oxidative phosphorylation and anaerobic respiration by acting as enzymes to catalyze these reactions or as other support proteins.

If the mitochondria's energy producing mechanisms fail to operate at an optimal level, overall cell function suffers due to a decline in the supply of available energy. Glucose may indeed be available for utilization by the mitochondria, but actual utilization rate of the glucose will be reduced if the cell's mitochondria are not functioning properly, with the result that the necessary supply of ATP molecules may not be adequate to supply the needs of the cell, thus limiting the function and survivability of the cell.

Creating a device to act as a means of correcting the cause of the decline in mitochondrial function would lead to increasing the body's utilization of glucose, which would result in a more optimal management of diabetes, obesity, the sense of fatigue some individuals experience, and the deleterious effects of the aging process.

Essential function of the mitochondrion relies on a variety of mRNAs being decoded by the ribosomes in order to the many produce proteins that act as enzymes and catalyze the biologic reactions of aerobic respiration. It does not appear that the mitochondrial DNA creates the majority of the required mRNAs to generate the enzymes required for aerobic respiration. The mRNAs are then either produced by the cell nucleus and sent to the mitochondria or the required mRNAs are created at the time a new mitochondrion is constructed. Loss of proper regulation of the chemical pathways inside the mitochondrion may be related to degradation of the necessary mRNAs. Since a significant portion of the population is obese and a significant portion of the population is diagnosed with diabetes, if the mRNAs do indeed degrade and are not adequately replenished, obesity and diabetes may be related to lack of proper function of mitochondria due to the fact that one or more mRNA molecules are not present in sufficient quantity to interact with ribosomes and thus not available to produce a sufficient quantity of the required enzymes needed to maintain the chemical pathways involved in the optimal metabolism of glucose.

A treatment of diabetes, obesity, chronic fatigue and aging may be approached by a medical device utilizing viruses as vehicles to transport biologically active mRNAs to mitochondria to bolster function of the mitochondria of all cells or possibly only certain cells in the body.

A Hepatitis C virus modified to carry a therapeutic mRNA payload could be introduced into the blood stream, travel the blood stream, engage the receptors on a liver cell with its surface probes, and then insert the genetic payload it carries into the liver cell. A genetic payload such as a quantity of messenger RNAs could be used to enhance proper protein production by cells deficient in a particular protein. In the case of a deficiency of a protein, production of a deficient protein could be enhanced by inserting a quantity of messenger RNAs into liver cells to stimulate production of the required protein. In the case of diabetes mellitus, utilizing a modified Hepatitis C virus as a vehicle, messenger RNA molecules could be inserted into liver cells, coded for any protein the liver cells are deficient in producing, with the intention of generating an adequate response to insulin and an optimal utilization of glucose.

The utilization of positive sense messenger RNA, does not permanently alter the cell's DNA. Messenger RNAs degrade and become unusable after a time. Use of RNA as a therapeutic modality offers a therapeutic opportunity that could have a reversible or an attenuatable effect when required. Exogenous messenger RNAs could begin producing the required proteins they are coded for as soon as they are inserted into a cell; where exogenous DNA would first need to be inserted into a nuclear chromosome, then read by a polymerase molecule in order to generate an endogenous messenger RNA that would be capable of stimulating the production of a desired protein. Messenger RNA also bypasses the action of decoding the DNA and errors or deficiencies that might occur during transcription phase in the nucleus. By employing a medically therapeutic virus to carry messenger RNA to cells, deficiencies of any of the approximately 30,000 proteins that comprise the tissues that exist in the body and on the surface of the body can be successfully treated or averted.

### BRIEF SUMMARY OF THE INVENTION

The medical device by which a modified virus or virus-like structure is used as a transport medium to carry a payload of one or more messenger ribonucleic acid (RNA) molecules to cells in the body. The modified virus or virus-like structure makes contact with a specific target cell by means of the virus's exterior probes or virus-like structure's exterior probes. Once the exterior probes engage the target cell's receptors, the modified virus or virus-like structure inserts into the target cell one or more messenger ribonucleic acid (mRNA) molecules it is carrying. Messenger RNA molecules inserted into the cell act similar to native messenger RNA molecules and either interact with the cell's ribosomes in the process of protein synthesis or interacts with the cell's native enzymes and undergoes further modification until the delivered messenger RNA molecule is capable of interacting with the cell's ribosomes in the process of protein synthesis. Medical disease states such as diabetes mellitus, obesity, chronic fatigue and aging, that are the result of a deficiency of one or more proteins, can be successfully treated by utilizing viruses or virus-like structures to insert the proper messenger RNA molecules into specific cells to enhance the production of proteins that are identified as being deficient, thus correcting the deficiency. Improved utilization of glucose and optimal production of energy molecules resulting from a robust and efficient metabolism of glucose will enhance the cells' capacity to carryout life sustaining functions and lead to a healthier individual.

### DETAILED DESCRIPTION

Diabetes mellitus, obesity, chronic fatigue syndrome, and aging may all be linked to the common pathway of degradation in an individual's capacity to metabolize glucose through the process of aerobic respiration. Errors in the DNA or errors that occur in the process that generates the messenger RNA or a deficiency in the number of messenger RNA or a deficiency in the number of biologically active messenger RNA results in a deficiency in cellular capacity to construct the enzymes involved in the biologic respiratory processes of glycolysis, tricarboxylic acid cycle and phosphorylation, which results in the medical conditions of diabetes mellitus, obesity, chronic fatigue and aging. Supplying the cells of the body with the means to produce sufficient quantities of biologically active enzymes to insure that the biologic respiratory processes of glycolysis, tricarboxylic acid cycle and oxidative phosphorylation occur at an optimal rate in cells would treat the medical conditions of diabetes mellitus, obesity, chronic fatigue and aging.

Viruses or virus-like structures can be fashioned to act as transport vehicles to carry and deliver messenger ribonucleic acid (mRNA) molecules to cells. The mRNA molecules carried by therapeutic viruses would supply the cells of the body with the genetic templates to construct the proteins the cell would be deficient in producing on its own.

Naturally occurring viruses can be altered by replacing the genetic material the virus would carry, with mRNA molecules that would have a beneficial medically therapeutic effect on cells. The naturally occurring virus would then carry and deliver to its natural target cell the payload of medically therapeutic mRNA molecules. As an example, Hepatitis viruses could be altered to carry medically therapeutic mRNA molecules to liver cells. The naturally occurring virus then, instead of causing disease associated with delivering its own genome to stimulate its own replication process, would instead act as a medical device to deliver a quantity of medically therapeutic mRNA molecules which would provide the target cell an enhanced capacity to generate proteins to carryout beneficial biologic processes to accomplish a medically therapeutic outcome.

Naturally occurring viruses can be further modified to have their naturally occurring glycoprotein surface probes replaced by glycogen surface probes that target specific cells in the body. Viruses modified to carry and deliver medically therapeutic mRNA molecules as the payload, further modified to have their glycoprotein surface probes, that cause the modified virus to engage specific cells in the body, provides a method whereby specific cells in the body can be targeted and this method embodies a means of providing to specific type of cell in the body an enhanced capacity to generate proteins to carryout beneficial biologic processes to accomplish a medically therapeutic outcome.

Virus-like structures can be constructed with similar physical characteristics to naturally occurring viruses and be fashioned to carry medically therapeutic mRNA molecules as the payload and have located on the surface glycoprotein probes that engage specific cells in the body. Viruses-like structures carrying medically therapeutic mRNA molecules as the payload, constructed to have their glycoprotein surface probes engage specific cells in the body, and deliver to those specific cells the mRNA the virus-like structures carry is a medical device whereby specific cells in the body can be targeted and this medical device embodies a means of providing to specific type of cell in the body an enhanced capacity to generate proteins to carryout beneficial biologic processes to accomplish a medically therapeutic outcome. The advantage of a virus-like structure is that the physical dimensions of the virus-like structure can be adjusted to accommodate variations in the physical size of the payload of medically therapeutic mRNA molecules, yet maintain a means of engaging targeted cells in the body and delivering to those targeted cells the mRNA molecules required to accomplish the desired medical therapeutic outcome. A second advantage of utilizing virus-like structures is to be able to change the surface characteristics of the transport vehicle to prevent the body's immune system from reacting to the presence of the therapeutic modified virus and destroying the modified virus before it is able to deliver the payload it carries to the cells it has been designed to target. HIV utilizes an exterior envelope comprised of the surface membrane of its host, the T-Helper cell, which acts as a disguise to fool the body's immune system detection resources. Virus-like structures could be fashioned, similar to HIV, to have as an exterior envelope a surface that resembles a cell's outer membrane in order to avoid detection by the body's immune system to improve survivability of the virus-like structure.

The Hepatitis C virus virion provides a naturally occurring specimen to illustrate the feasibility of the medical device described in this text. The Hepatitis C virus (HCV) virion is comprised of an outer lipoprotein envelope and an internal nucleocapsid. The virus's genetic payload is carried within its core, the nucleocapsid. The HCV's naturally occurring genetic payload consists of a single molecule of linear positive sense, single stranded RNA approximately 9600 nucleotides in length, which includes: the structural proteins of C, E1, E2, the nonstructural proteins NS1, NS2, NS3, NS4A, NS4B, NS5A, NS5B, p7 and ARFP/F protein. Present on the surface of the outer envelope of the Hepatitis C virus virion are probes that detect receptors present on the surface of liver cells. The glycoproteins E1 and E2 have been identified to be affixed to the surface of HCV virion. Portions of the Hepatitis C virus genome, when separated into individual pieces, behave like messenger RNA. The naturally occurring HCV virion is constructed with surface probes fashioned to recognize receptors on the surface of a liver cell. Once the naturally occurring HCV's surface probe E2 engages a liver cell's CD81 receptor, and cofactors on the surface of HCV's exterior envelope engage the low density lipoprotein receptor (LDLR) or the scavenger receptor class B type I (SR-BI) on the liver cell, the HCV virion then has the opportunity to insert its RNA genetic payload into the engaged target liver cell.

The Hepatitis C virus virion carrying a mRNA payload, infects liver cells with its payload for the purpose of causing the now infected cell to generate a variety of proteins that will be assembled into copies resembling the original HCV virion. The copies of the HCV virion are then released from the infected cell to migrate in search of other host cells. Variations in the Hepatitis C virus are based on variations that occur in the strand of mRNA molecule the HCV virion carries as it genome. HCV virions may therefore carry differing mRNA molecules as its genetic payload and deliver these mRNA molecules to specific liver cells in the body to cause these cells to produce proteins to accomplish the task of replication of similar HCV virions.

Replicating viruses and constructing viruses to carry DNA payloads is a form of manufacturing technology that has already been well established and is in use facilitating the concept of gene therapy. Replicating viruses and designing these viruses to carry messenger ribonucleic acid as the genetic payload would incorporate similar techniques as already proven useful in current DNA gene therapy technologies.

To carry out the process to manufacture a modified medically therapeutic Hepatitis C virus, messenger RNA that would code for the general physical outer structures of the Hepatitis C virus would be inserted into a host. The host may include devices such as a host cell or a hybrid host cell. The host may utilize DNA or RNA or a combination of genetic instructions in order to accomplish the construction of medically therapeutic modified virus virions. The DNA or messenger RNA molecules to create the medically therapeutic hepatitis virus would direct the cells to generate copies of the medically therapeutic virus carrying a medically therapeutic mRNA payload. In some cases DNA or messenger RNA would be inserted into the host that would be coded to cause the production of surface probes that would be affixed to the surface of the virus virion that would target the surface receptors on specific cells in the body other than the liver cells the Hepatitis C virus naturally targets. DNA or messenger RNA would direct the host to generate copies of the messenger RNA that would provide a therapeutic action, this medically therapeutic messenger RNA would take the place of the Hepatitis C virus's innate genome as its payload. The medical treatment form of the Hepatitis C virus carrying the medically therapeutic messenger RNA would be produced, assembled and released from a host. Virus-like structures would be generated in similar fashion using a host such as host-cells or hybrid host cells. The copies of the medically therapeutic hepatitis virus or virus-like structures, upon exiting the host, would be collected, stored and utilized as a medical treatment as necessary.

To treat the various different forms of diabetes mellitus various combinations of messenger RNA would be inserted into the host, and the host would produce copies of modified Hepatitis C virus that target liver cells and carry a genetic payload consisting of messenger RNA molecules that would consist of one or more copies of a messenger RNA that codes for the insulin receptor, the enzymes utilized in the processes of glucose metabolism including glycolysis, tricarboxylic acid cycle and oxidative phosphorylation. Depending upon the physical size of the messenger RNAs and the available space inside the modified Hepatitis C virus more than one type of messenger RNA may be packaged into a single modified Hepatitis C virus, which would produce one or more therapeutic actions in a cell. In some cases enzymes that catalyze the chemical reactions in aerobic and anaerobic respiration pathways are comprised of more than one protein molecule and would require the action of several mRNA molecules to create the physical entity of the enzyme in its entirety.

The modified Hepatitis C virus and virus-like structures would be incapable of replication on its own due to the fact that the messenger RNA that would code for the replication process to produce copies of the modified virus or virus-like structure would not be present in the modified form of the Hepatitis C virus or virus-like structure. The modified form of the Hepatitis C virus or virus-like structure would carry messenger RNA that would be coded for generating a variety of enzymes that would produce a medically therapeutic and beneficial result. Enzymes such messenger RNA would code for would include the enzymes listed in the following paragraphs.

Hexokinase (EC 2.7.1.1) also referred to as hexokinase type IV glucokinase or simply glucokinase. Hexokinase converts glucose to glucose-6-phosphate in the process of glycolysis.

Glucose-6-phosphate isomerase (EC 5.3.1.9) also known as glucose-6-phosphate isomerase. Glucose-6-phosphate isomerase is an enzyme that converts glucose-6-phosphate to fructose-6-phosphate in the process of glycolysis.

Phosphofructokinase (EC 2.7.1.11) also known as 6-phosphofructokinase. Phosphofructokinase is an enzyme that converts fructose-6-phosphate to Fructose 1,6-diphosphate in the process of glycolysis.

Fructose bisphosphate aldolase (EC 4.1.2.13), also known as aldolase. Fructose bisphosphate aldolase is an enzyme that converts fructose 1,6-diphosphate to two different entities including dihydroxyacetone 3-phosphate and glyceraldehydes 3-phosphate in the process of glycolysis.

Triose-phosphate dehydrogenase (EC 5.3.1.1). Triose-phosphate dehydrogenase is an enzyme that converts glyceraldehydes 3-phosphate to 1,3-diphosphoglycerate in the process of glycolysis.

Phosphoglycerate kinase (EC 2.7.2.3). Phosphoglycerate kinase is an enzyme that converts 1,3-diphosphoglycerate to 3-phosphoglycerate in the process of glycolysis.

Phosphoglycerate mutase (EC 5.4.2.1). Phosphoglycerate mutase is an enzyme that converts 3-phosphoglycerate to 2-phosphoglycerate in the process of glycolysis.

Enolase (EC 4.2.1.11). Enolase is an enzyme that converts 2-phosphoglycerate to phosphoenolpyruvate in the process of glycolysis.

Pyruvate kinase (EC 2.7.2.3). Pyruvate kinase is an enzyme that converts phosphoenolpyruvate to pyruvate in the process of glycolysis.

Pyruvate dehydrogenase is comprised of three units. The three units include E1 (EC 1.2.4.1), (EC 1.2.1.51), E2 dihydrolipoamide S-acetyltransferase (EC 2.3.1.12), and E3 dihydrolipoamide dehydrogenase (EC 1.8.1.4). Pyruvate dehydrogenase molecular complex catalyzes the conversion of pyruvate to acetyl-CoA.

Citrate synthetase (EC 4.1.3.7). Citrate synthetase is an enzyme that converts acetyl-CoA combines with oxaloacetate to produce citrate in the tricarboxylic acid cycle.

Aconitase (EC 4.2.1.3). Aconitase is an enzyme that converts citrate to cis-aconitate in the tricarboxylic acid cycle. Aconitase is an enzyme that converts cis-aconitate to iso-citrate in the tricarboxylic acid cycle.

Isocitrate dehydrogenase (EC 1.1.1.41). Isocitrate dehydrogenase is an enzyme that converts isocitrate to alpha-ketoglutaric acid in the tricarboxylic acid cycle.

2-oxoglutarate dehydrogenase is protein complex comprised of three units. The three units include E1 (EC 1.2.4.2), E2 (EC 2.3.1.61), and E3 (EC 1.8.1.4). 2-oxoglutarate dehydrogenase is an enzyme complex that converts alpha-ketoglutaric to succinyl-CoA in the tricarboxylic acid cycle.

Succinyl-CoA synthetase (EC 6.2.1.5). Succinyl-CoA synthetase is an enzyme that converts succinyl CoA to succinate in the tricarboxylic acid cycle.

Succinate dehydrogenase (EC 1.3.5.1). Succinate dehydrogenase is an enzyme that converts succinate to fumarate in the tricarboxylic acid cycle.

Fumarate hydratase (EC 4.2.1.2). Fumarate hydratase is an enzyme that converts fumarate to malate in the tricarboxylic acid cycle.

Malate dehydrogenase (EC 1.1.1.37). Malate dehydrogenase is an enzyme that converts malate to oxaloacetate in the tricarboxylic acid cycle.

NADH dehydrogenase (EC 1.6.5.3) molecule, also referred to as NADH-coenzyme Q oxidoreductase or complex 1, is utilized in oxidative phosphorylation.

Succinate dehydrogenase (EC 1.3.5.1) molecule, also referred to as succinate oxidoreductase or complex II, is utilized in oxidative phosphorylation.

Cytochrome-c reductase (EC 1.10.2.2) molecule, also referred to as complex III, is utilized in oxidative phosphorylation.

Cytochrome-c oxidase (EC 1.9.3.1) molecule, also referred to as complex IV, is utilized in oxidative phosphorylation.

ATP synthase (EC 3.6.1.34) molecule is utilized in oxidative phosphorylation.

Lactate dehydrogenase (EC 1.1.1.27) molecule is utilized to convert pyruvate to lactic acid in anaerobic respiration.

In review, the medical device described in this text includes taking a naturally occurring virus and altering its payload so that it transports medically therapeutic messenger RNA to cells it was naturally designed to infect, but instead of delivering its own genetic payload, it delivers the medically therapeutic messenger RNA it is carrying so that those cells may benefit from being able to produce protein molecules the messenger RNA are coded for, and the medical device described in this text includes taking a naturally occurring virus and altering its payload so that it carries medically therapeutic messenger RNA to cells and alter the virus's glycoprotein probes so that it is capable of infecting specifically targeted cells, but instead of delivering its own genetic payload, it delivers to specific cells the medically therapeutic messenger RNA it is carrying so that those cells may benefit from being able to produce protein molecules the messenger RNA is coded for, and the medical device described in this text includes taking a virus-like structure, which carries medically therapeutic messenger RNA to cells, affixed to the surface glycoprotein probes so that it is capable of delivering medically therapeutic messenger RNA it is carrying to specific target cells so that those cells may benefit from being able to produce protein molecules the messenger RNA is coded for once the messenger RNA is delivered to the specific cells. Diabetes mellitus, obesity, chronic fatigue, and aging may all be linked to the vital process of glucose metabolism and aerobic respiration. Providing the body with the means of maintaining an optimum level of glucose metabolism and conducting aerobic respiration, in some cases anaerobic respiration, may provide the means to effectively manage the above-mentioned four major health conditions.

As mentioned above, the medical device to improve the current glucose management to enhance the treatment diabetes mellitus, a quantity of modified virus virions, such as modified Hepatitis C virus virions or virus-like structures would be introduced into a patient's blood stream or tissues so that the modified virus could deliver the medically therapeutic mRNA payload that it carries to targeted cells in the body, such as liver cells. Once the modified virus virions insert their medically therapeutic payload consisting of messenger RNA into the cell the modified virus virion has been targeted for the cell's biologic function of producing insulin receptors or metabolizing glucose by way of glycolysis, tricarboxylic acid cycle, oxidative phosphorylation or anaerobic respiration are to be enhanced. Improvement in the metabolism of glucose inside cells will reduce circulating levels of glucose in the blood stream and decrease a patient's likelihood of diabetes mellitus.

As mentioned above, the medical device to improve the current glucose management to enhance the treatment obesity, a quantity of modified virus virions, such as modified Hepatitis C virus virions or virus-like structures, would be introduced into a patient's blood stream or tissues so that the modified virus could deliver the medically therapeutic mRNA payload that it carries to targeted cells in the body, such as liver cells. Once the modified virus virions insert their medically therapeutic payload consisting of messenger RNA into the cell the modified virus virion has been targeted for, the cell's biologic function of producing insulin receptors or metabolizing glucose by way of glycolysis, tricarboxylic acid cycle, oxidative phosphorylation or anaerobic respiration are to be enhanced. Improvement in the metabolism of glucose inside cells will reduce circulating levels of glucose in the blood stream and decrease a patient's likelihood of obesity by increasing the cellular consumption of glucose and fats.

As mentioned above, the medical device to improve the current glucose management so as to enhance the treatment chronic fatigue, a quantity of modified virus virions, such as modified Hepatitis C virus virions or virus-like structures, would be introduced into a patient's blood stream or tissues so that the modified virus could deliver the medically therapeutic mRNA payload that it carries to targeted cells in the body, such as muscle cells. Once the modified virus virions insert their medically therapeutic payload consisting of messenger RNA into the targeted cells, the cells' biologic function of producing insulin receptors or metabolizing glucose by way of glycolysis, tricarboxylic acid cycle, oxidative phosphorylation or anaerobic respiration are to be enhanced. Improvement in the metabolism of glucose inside cells will increase the production of ATP, the result of which will be increasing the available energy resources cells have to conduct biologic processes inside the cell, which will lead to less fatigue in patients.

As mentioned above, the medical device to improve the current glucose management so as to enhance the treatment aging, a quantity of modified virus virions, such as modified Hepatitis C virus virions or virus-like structures, would be introduced into a patient's blood stream or tissues so that the modified virus could deliver the medically therapeutic mRNA payload that it carries to targeted cells in the body, such as chondrocytes, cells responsible for the production of cartilage on the joint surfaces of bone. Once the modified virus virions insert their medically therapeutic payload consisting of messenger RNA into the targeted cells, the cells' biologic function of producing insulin receptors or metabolizing glucose by way of glycolysis, tricarboxylic acid cycle, oxidative phosphorylation or anaerobic respiration are to be enhanced. Improvement in the metabolism of glucose inside cells will increase the production of ATP, the result of which will be increasing the available energy resources cells have to conduct biologic processes inside the cell, which will lead to a decline in the failure rate of cells in the body which will forestall and may reverse the process of aging in patients.

By utilizing the described medical device to provide the cells of the body with the above-mentioned medically therapeutic messenger RNA molecules and enhancing the capacity of cells to carry out the biologic processes of glycolysis, tricarboxylic acid cycle, oxidative phosphorylation and anaerobic respiration, will result in an efficient means to control the glucose levels in the blood stream and utilize glucose to produce energy molecules such as ATP molecules in the cells of the body, such as liver cells, on a constant and persistent basis by utilizing innate regulatory mechanisms, will result in effectively managing diabetes mellitus, obesity, chronic fatigue and aging for the betterment of the medical care of patients.

### DRAWING

None.

What is claimed:

1. A medical device for inserting a quantity of messenger ribonucleic acid molecules into cells of the body comprising:
(a) a quantity of modified virus virions generated for the purpose of transporting a quantity of said messenger ribonucleic acid molecules,
(b) the quantity of said modified virus virions having a quantity of glycoprotein probes affixed to their surface, said glycoprotein probes constructed in a manner to target specific cells in said body,
(c) said glycoprotein probes capable of engaging specific cell-surface receptors on said cells,
(d) once said glycoprotein probes have successfully engaged cell-surface receptors on said cell, the quantity of said modified virus virions deliver into said cells a quantity of said messenger ribonucleic acid molecules said modified virus virions are carrying,
whereby said cellular structures are intended to read and decipher the biologic instructions coded into the quantity of said messenger ribonucleic acid molecules and construct said proteins,
whereby said cells will be made capable of producing said protein molecules by cellular structures, for purposes of accomplishing a medical treatment,
whereby said cells' biochemistry capacity will be improved regarding producing said protein molecules by said cellular structures, for purposes of accomplishing a medical treatment.

2. The medical device in claim 1 wherein said modified virus virions selected from the group consisting of naturally occurring virus virions whose payload has been altered to carry a quantity of messenger ribonucleic acid molecules, naturally occurring virus virions whose payload has been altered to carry a quantity of messenger ribonucleic acid molecules said virus virions capable of delivering said quantity of messenger ribonucleic acid molecules which the surface glycoprotein probes have been altered in a manner the glycoprotein probes are fashioned to engage specific cells in said body, and virus-like structures constructed to resemble naturally occurring virus virions said virus-like structures capable of carrying a quantity of messenger ribonucleic acid molecules said virus-like structures constructed with glycoprotein probes fashioned to engage specific cells in said body said virus-like structures capable of delivering said quantity of messenger ribonucleic acid molecules to said specific cells in said body.

3. The medical device in claim 1 wherein said cellular structures are ribosomes.

4. The medical device in claim 1 wherein said body is comprised of the physical features of the human body.

5. A medical device for inserting a quantity of messenger ribonucleic acid molecules into cells of the body comprising:
(a) a quantity of modified virus virions generated for the purpose of transporting a quantity of said messenger ribonucleic acid molecules,
(b) the quantity of said modified virus virions having a quantity of glycoprotein probes affixed to their surface, said glycoprotein probes constructed in a manner to target specific cells in said body,
(c) said glycoprotein probes capable of engaging specific cell-surface receptors on said cells,
(d) once said glycoprotein probes have successfully engaged cell-surface receptors on said cell, the quantity of said modified virus virions deliver into said cells a quantity of said messenger ribonucleic acid molecules said modified virus virions are carrying,
whereby said cellular structures are intended to read and decipher the biologic instructions coded into the quantity of said messenger ribonucleic acid molecules and construct proteins,
whereby said cells will be made capable of producing said protein molecules by cellular structures, for purposes of accomplishing a medical treatment,
whereby said cells' biochemistry capacity will be improved regarding producing said protein molecules by said cellular structures, for purposes of accomplishing a medical treatment to enhance deficiencies in the biologic processes of aerobic respiration which results in the production of energy molecules,
whereby said cells' biochemistry capacity will be improved regarding producing protein molecules by cellular structures, for purposes of accomplishing a medical treatment to enhance deficiencies in the biologic processes of anaerobic respiration which results in the production of energy molecules.

6. The medical device in claim 5 wherein said modified virus virions selected from the group consisting of naturally occurring virus virions whose payload has been altered to carry a quantity of messenger ribonucleic acid molecules, naturally occurring virus virions whose payload has been altered to carry a quantity of messenger ribonucleic acid molecules said virus virions capable of delivering said quantity of messenger ribonucleic acid molecules which the surface glycoprotein probes have been altered in a manner the glycoprotein probes are fashioned to engage specific cells in said body, and virus-like structures constructed to resemble naturally occurring virus virions said virus-like structures capable of carrying a quantity of messenger ribonucleic acid molecules said virus-like structures constructed with glycoprotein probes fashioned to engage specific cells in said body said virus-like structures capable of delivering said quantity of messenger ribonucleic acid molecules to said specific cells in said body.

7. The medical device in claim 5 wherein said cellular structures are ribosomes.

8. The medical device in claim 5 wherein said messenger ribonucleic acid molecules selected from the group consisting of those messenger ribonucleic acid molecules that produce enzymes that participate in the biologic process of glycolysis, those messenger ribonucleic acid molecules that produce enzymes that participate in the biologic process of the tricarboxylic acid cycle, those messenger ribonucleic acid molecules that produce enzymes that participate in the biologic process of oxidative phosphorylation, and those messenger ribonucleic acid molecules that produce enzymes that participate in the biologic process of anaerobic respiration.

9. The medical device in claim 5 wherein said specific cells selected from the group consisting of cells comprising the muscles, cells comprising the brain, cells comprising the heart, cells comprising the pancreas, cells comprising the endocrine glands, cells comprising the dermis, cells comprising the mucosa, cells comprising the gastroenteric tract, cells comprising the renal system, cells comprising the skeletal structures, cells comprising the pulmonary system, cells comprising the nervous system, cells comprising the immune system, cells comprising the sex organs, cells comprising the connective tissues, cells comprising the spleen, cells comprising the eyes, cells comprising the reticuloendothelial system, and cells comprising the liver.

10. The medical device in claim 5 wherein said quantity of said messenger ribonucleic acid molecules code for subunits of an enzyme molecule, whereby the decoding of a quantity of differing messenger ribonucleic acid molecules by said cellular structures will result in the construction of said enzyme molecule in its entirety.

11. The medical device in claim 5 wherein said body is comprised of the physical features of the human body.

12. The medical device in claim 5 wherein said messenger ribonucleic acid molecules selected from the group consisting of
messenger ribonucleic acid molecules to code for the insulin receptor molecule,
messenger ribonucleic acid molecules to code for the hexokinase Enzyme Commission of the International Union of Biochemistry (EC) 2.7.1.1 enzyme molecule,
messenger ribonucleic acid molecules to code for the glucose-6-phosphophate isomerase EC 5.3.1.9 enzyme molecule,
messenger ribonucleic acid molecules to code for the phosphofructo kinase EC 2.7.1.11 enzyme molecule,
messenger ribonucleic acid molecules to code for the fructose bisphosphate aldolase EC 4.1.2.13 enzyme molecule,
messenger ribonucleic acid molecules to code for the triose-phosphate isomerase EC 5.3.1.1 enzyme molecule,
messenger ribonucleic acid molecules to code for the glyceraldehyde-3-phosphate dehydrogenase EC 1.2.1.12 enzyme molecule,
messenger ribonucleic acid molecules to code for the phosphoglycerate kinase EC 2.7.2.3 enzyme molecule,
messenger ribonucleic acid molecules to code for the phosphoglycerate mutase EC 5.4.2.1 enzyme molecule,
messenger ribonucleic acid molecules to code for the enolase EC 4.2.1.11 enzyme molecule,
messenger ribonucleic acid molecules to code for the pyruvate kinase EC 2.7.1.40 enzyme molecule,
messenger ribonucleic acid molecules to code for the pyruvate dehydrogenase (acetyl-transferring) EC 1.2.4.1 enzyme molecule,
messenger ribonucleic acid molecules to code for the pyruvate dehydrogenase (NADP+) EC 1.2.1.51 enzyme molecule,
messenger ribonucleic acid molecules to code for the dihydrolipoamide S-acetyltransferase EC 2.3.1.12 enzyme molecule,
messenger ribonucleic acid molecules to code for the dihydrolipoamide dehydrogenase EC 1.8.1.4 enzyme molecule,
messenger ribonucleic acid molecules to code for the citrate synthetase EC 4.1.3.7 enzyme molecule,
messenger ribonucleic acid molecules to code for the aconitase EC 4.2.1.3 enzyme molecule,
messenger ribonucleic acid molecules to code for the isocitrate dehydrogenase EC 1.1.1.41 enzyme molecule,
messenger ribonucleic acid molecules to code for the 2-oxoglutarate dehydrogenase E1 EC 1.2.4.2 enzyme molecule,
messenger ribonucleic acid molecules to code for the 2-oxoglutarate dehydrogenase E2 EC 2.3.1.61 enzyme molecule,
messenger ribonucleic acid molecules to code for the 2-oxoglutarate dehydrogenase E3 EC 1.8.1.4 enzyme molecule,
messenger ribonucleic acid molecules to code for the succinyl-CoA synthetase EC 6.2.1.5 enzyme molecule,
messenger ribonucleic acid molecules to code for the succinate dehydrogenase EC 1.3.5.1 enzyme molecule,
messenger ribonucleic acid molecules to code for the fumarate hydratase EC 4.2.1.2 enzyme molecule,
messenger ribonucleic acid molecules to code for the malate dehydrogenase EC 1.1.1.37 enzyme molecule,
messenger ribonucleic acid molecules to code for the nicotinamide adenine dinucleotide (NADH) dehydrogenase EC 1.6.5.3 enzyme molecule,
messenger ribonucleic acid molecules to code for the succinate dehydrogenase EC 1.3.5.1 enzyme molecule,
messenger ribonucleic acid molecules to code for the cytochrome-c reductase EC 1.10.2.2 enzyme molecule,
messenger ribonucleic acid molecules to code for the cytochrome-c oxidase EC 1.9.3.1 enzyme molecule,
messenger ribonucleic acid molecules to code for the ATP synthase EC 3.6.1.34 enzyme molecule,
messenger ribonucleic acid molecules to code for the lactate dehydrogenase EC 1.1.1.27 enzyme molecule,
messenger ribonucleic acid molecules to code for the adenosine triphosphate molecule,
messenger ribonucleic acid molecules to code for the adenosine diphosphate molecule,
messenger ribonucleic acid molecules to code for the nicotinamide adenine dinucleotide molecule, and
messenger ribonucleic acid molecules to code for the flavin adenine dinucleotide molecule.

13. The medical device in claim 12 wherein said enzyme molecule is comprised of a quantity of protein subunits to create a protein complex.

14. A medical device for inserting a quantity of messenger ribonucleic acid molecules into liver cells of the body comprising:
(a) a quantity of modified virus virions generated for the purpose of transporting a quantity of said messenger ribonucleic acid molecules,
(b) the quantity of said modified virus virions having a quantity of glycoprotein probes affixed to their surface, said glycoprotein probes constructed in a manner to target said liver cells in said body,
(c) said glycoprotein probes capable of engaging specific cell-surface receptors on said liver cells,
(d) once said glycoprotein probes have successfully engaged cell-surface receptors on said liver cells, the quantity of said modified virus virions deliver into said liver cells a quantity of said messenger ribonucleic acid molecules said modified virus virions are carrying,
whereby said cellular structures are intended to read and decipher the biologic instructions coded into the quantity of said messenger ribonucleic acid molecules and construct said proteins,
whereby said liver cells will be made capable of producing protein molecules by cellular structures, for purposes of accomplishing a medical treatment,
whereby said liver cells' biochemistry capacity will be improved regarding producing protein molecules by cellular structures, for purposes of accomplishing a medical treatment to enhance deficiencies in the biologic processes of aerobic respiration which results in the production of energy molecules,
whereby said liver cells' biochemistry capacity will be improved regarding producing protein molecules by cellular structures, for purposes of accomplishing a medical treatment to enhance deficiencies in the biologic processes of anaerobic respiration which results in the production of energy molecules.

15. The medical device in claim 14 wherein said cellular structures are ribosomes.

16. The medical device in claim 14 wherein said protein selected from the group consisting of those enzymes that participate in the biologic process of glycolysis, those enzymes that participate in the biologic process of tricarboxylic acid cycle, those enzymes that participate in the biologic process of oxidative phosphorylation and those enzymes that participate in the biologic process of anaerobic respiration.

17. The medical device in claim 14 wherein said modified virus virions selected from the group consisting of Hepatitis A virus virions, Hepatitis B virus virions, Hepatitis C virus virions, Hepatitis D virus virions, Hepatitis F virus virions, Hepatitis E virus virions, Hepatitis G virus virions, and Hepatitis H virus virions.

18. The medical device in claim 14 wherein said quantity of said messenger ribonucleic acid molecules code for subunits of an enzyme molecule, whereby the decoding of a quantity of differing messenger ribonucleic acid molecules by said cellular structures will result in the construction of said enzyme molecule in its entirety.

19. The medical device in claim 14 wherein said body is comprised of the physical features of the human body.

20. The medical device in claim 14 wherein said messenger ribonucleic acid molecules selected from the group consisting of
messenger ribonucleic acid molecules to code for the insulin receptor molecule,
messenger ribonucleic acid molecules to code for the hexokinase Enzyme Commission of the International Union of Biochemistry (EC) 2.7.1.1 enzyme molecule,
messenger ribonucleic acid molecules to code for the glucose-6-phosphophate isomerase EC 5.3.1.9 enzyme molecule,
messenger ribonucleic acid molecules to code for the phosphofructo kinase EC 2.7.1.11 enzyme molecule,
messenger ribonucleic acid molecules to code for the fructose bisphosphate aldolase EC 4.1.2.13 enzyme molecule,
messenger ribonucleic acid molecules to code for the triose-phosphate isomerase EC 5.3.1.1 enzyme molecule,
messenger ribonucleic acid molecules to code for the glyceraldehyde-3-phosphate dehydrogenase EC 1.2.1.12 enzyme molecule,
messenger ribonucleic acid molecules to code for the phosphoglycerate kinase EC 2.7.2.3 enzyme molecule,
messenger ribonucleic acid molecules to code for the phosphoglycerate mutase EC 5.4.2.1 enzyme molecule,
messenger ribonucleic acid molecules to code for the enolase EC 4.2.1.11 enzyme molecule,
messenger ribonucleic acid molecules to code for the pyruvate kinase EC 2.7.1.40 enzyme molecule,
messenger ribonucleic acid molecules to code for the pyruvate dehydrogenase (acetyl-transferring) EC 1.2.4.1 enzyme molecule,
messenger ribonucleic acid molecules to code for the pyruvate dehydrogenase (NADP+) EC 1.2.1.51 enzyme molecule,
messenger ribonucleic acid molecules to code for the dihydrolipoamide S-acetyltransferase EC 2.3.1.12 enzyme molecule,
messenger ribonucleic acid molecules to code for the dihydrolipoamide dehydrogenase EC 1.8.1.4 enzyme molecule,
messenger ribonucleic acid molecules to code for the citrate synthetase EC 4.1.3.7 enzyme molecule,
messenger ribonucleic acid molecules to code for the aconitase EC 4.2.1.3 enzyme molecule,
messenger ribonucleic acid molecules to code for the isocitrate dehydrogenase EC 1.1.1.41 enzyme molecule,
messenger ribonucleic acid molecules to code for the 2-oxoglutarate dehydrogenase E1 EC 1.2.4.2 enzyme molecule,
messenger ribonucleic acid molecules to code for the 2-oxoglutarate dehydrogenase E2 EC 2.3.1.61 enzyme molecule,
messenger ribonucleic acid molecules to code for the 2-oxoglutarate dehydrogenase E3 EC 1.8.1.4 enzyme molecule,
messenger ribonucleic acid molecules to code for the succinyl-CoA synthetase EC 6.2.1.5 enzyme molecule,
messenger ribonucleic acid molecules to code for the succinate dehydrogenase EC 1.3.5.1 enzyme molecule,
messenger ribonucleic acid molecules to code for the fumarate hydratase EC 4.2.1.2 enzyme molecule,
messenger ribonucleic acid molecules to code for the malate dehydrogenase EC 1.1.1.37 enzyme molecule,
messenger ribonucleic acid molecules to code for the nicotinamide adenine dinucleotide (NADH) dehydrogenase EC 1.6.5.3 enzyme molecule,
messenger ribonucleic acid molecules to code for the succinate dehydrogenase EC 1.3.5.1 enzyme molecule,
messenger ribonucleic acid molecules to code for the cytochrome-c reductase EC 1.10.2.2 enzyme molecule,
messenger ribonucleic acid molecules to code for the cytochrome-c oxidase EC 1.9.3.1 enzyme molecule,
messenger ribonucleic acid molecules to code for the ATP synthase EC 3.6.1.34 enzyme molecule,
messenger ribonucleic acid molecules to code for the lactate dehydrogenase EC 1.1.1.27 enzyme molecule,
messenger ribonucleic acid molecules to code for the adenosine triphosphate molecule,
messenger ribonucleic acid molecules to code for the adenosine diphosphate molecule,
messenger ribonucleic acid molecules to code for the nicotinamide adenine dinucleotide molecule, and
messenger ribonucleic acid molecules to code for the flavin adenine dinucleotide molecule.
